(19) United States
(12) Patent Application Publication
(10) Pub. No.: US 2025/0115959 A1
(43) Pub. Date: Apr. 10, 2025

(54) METHOD FOR SCREENING PRE-IMPLANTATION EMBRYOS

(71) Applicant: GUANGZHOU NVWA LIFE TECHNOLOGY CO., LTD, Guangzhou, Guangdong (CN)

(72) Inventors: Jiang LIU, Guangzhou (CN); Zi-jiang CHEN, Guangzhou (CN); Yuan GAO, Guangzhou (CN); Xuelong YAO, Guangzhou (CN); Lizhi YI, Guangzhou (CN)

(21) Appl. No.: 18/567,661

(22) PCT Filed: Nov. 17, 2022
(86) PCT No.: PCT/CN2022/132461

§ 371 Date: Dec. 6, 2023

(30) Foreign Application Priority Data
Sep. 26, 2022 (CN) 202211173465.8

(51) Int. Cl. C12Q 1/6883; C12Q 1/6874

(52) U.S. Cl. CPC C12Q 1/6883; C12Q 1/6874; C12Q 2600/118; C12Q 2600/154

(57) ABSTRACT

The present application relates to a method for screening pre-implantation embryos. The screening method includes steps of testing chromosomes and DNA methylation levels of the pre-implantation embryos; and selecting, from the pre-implantation embryos according to the obtained test result, the embryo having normal chromosomes, a DNA methylation level of a target region meeting preset requirements, and a genome-wide DNA methylation level within an interval of about 0.25 to 0.27 or a genome-wide DNA methylation level outside the interval of about 0.25 to 0.27 but close to the interval of about 0.25 to 0.27 relative to other embryos as the embryo to be implanted.

### RELATED APPLICATIONS

This application claims priority to Chinese patent application No. 2022111734658, filed on Sep. 26, 2022 and entitled “Method for screening pre-implantation embryos”, the content of which is hereby incorporated by reference in its entirety.

### TECHNICAL FIELD

The present application relates to the technical field of assisted reproductive medicine, in particular to a method for screening pre-implantation embryos.

### BACKGROUND

Assisted reproductive technology is an effective clinical technology to solve infertility. According to the statistics of “Reproductive Biology and Endocrinology”, more than 20% of infertile couples have to be treated by the assisted reproductive technology.

Improving the birth rate of assisted reproduction has always been a challenge in the field of assisted reproductive medicine. An effective way to increase the birth rate is to select excellent embryos for implantation in the process of assisted reproduction. Currently, morphological indicators and preimplantation genetic screening (PGS) are commonly used in clinic to evaluate and select embryos.

Using morphological indicators to evaluate and select embryos mainly refers to scoring the generated blastocysts, according to the Gardner blastocyst scoring system, as for example 4AA, 4BB, 4AB, etc. For the score, the number represents the density of blastocyst cells, and the grade of the degree of division, including levels 1 to 6, with 6 being the best. The first letter represents the quality of the inner cell mass that will develop into a fetus, and the second letter represents the quality of trophoblast cells that will develop into a placenta, with the best quality grade as A, followed by B and C.

Preimplantation genetic screening (PGS) of an embryo refers to a method for measuring the number and structure of chromosomes of the embryo and determining whether the embryo has abnormal genetic material through biopsies of 3 to 5 embryo cells before implantation. PGS technology has been developed for more than 30 years, from the initial use of fluorescence in situ hybridization (FISH) to analyze the ploidy and structure of a few specific chromosomes only, to the current use of whole genome amplification (WGA) to achieve the testing of 23 pairs of chromosomes. Although using morphological indicators to screen embryos is easy to operate, the morphological indicators cannot reflect whether the embryo has abnormal genetic material, and cannot reflect the developmental quality of the embryo and the risk of carrying diseases. A study revealed that only 42% of embryos were chromosomally completely normal, and only 30% of embryos with the inner cell mass (ICM) rated as grade A were chromosomally normal among the blastocysts with good morphology (Munne et al., 2010). Clinical studies have discovered that the probability of embryonic chromosomal abnormalities will increase with the age of parents, especially the age of the female. Studies further found that the incidence rate of aneuploidy in oocytes and embryos gradually increases (34% to 75%) after 35 years old (Franasiak et al., 2014). Therefore, the probability of natural pregnancy and in-vitro fertilization (IVF) pregnancy significantly decreased and the abortion rate increased for elderly women (Ola and Li, 2006). The efficiency of morphological screening of embryos is further reduced for the elderly population. Therefore, embryos in some elderly women patients aged over 35 years old and having recurrent abortion or repeated implantation failures were further screened by the PGS technology (Chen et al., 2015; Rubio et al., 2017).

The PGS technology has improved the birth rate of assisted reproduction to a certain extent, and the birth rate of test-tube babies has reached 40% after PGS screening. However, clinical studies indicated that substantial embryos with normal chromosome ploidy cannot be successfully conceived. Recent studies show that PGS has no significant effect on improving pregnancy rates for female patients aged below 35 years (Yan et al., 2021). On the other hand, the incidence rate of epigenetic diseases in test-tube babies is twice as high as the incidence rate in naturally conceived babies, with about 1 in 1,000 babies suffering from imprinted gene diseases. The PGS technology can test only the genomic DNA of embryos but cannot test the epigenetic DNA of embryos, so PGS cannot determine whether the embryo has an epigenetic disease.

Based on this, the pregnancy rate is low with the current pre-implantation test method in clinic.

In view of above, the present application is proposed.

### SUMMARY

Accordingly, the embodiments of the present application provide a method for screening pre-implantation embryos. The present application provides a technical solution as follows.

The present application provides a method for screening pre-implantation embryos, the method including:
testing chromosomes and DNA methylation levels of the pre-implantation embryos, and
selecting, from the pre-implantation embryos according to the obtained test result, the embryo having normal chromosomes, and a genome-wide DNA methylation level within an interval of about 0.25 to 0.27 or a genome-wide DNA methylation level outside the interval of about 0.25 to 0.27 but close to the interval of about 0.25 to 0.27 relative to other embryos as the embryo to be implanted.

In some embodiments of the present application, the pre-implantation embryos are embryos that have developed in vitro for about 5 to 7 days after fertilization.

In some embodiments of the present application, the pre-implantation embryos are embryos at a blastocyst stage.

In some embodiments of the present application, a sample for testing the chromosomes and the DNA methylation level includes a trophoblast cell from the pre-implantation embryo.

In some embodiments of the present application, the normal chromosomes include chromosomes with a normal number and a normal structure, or chromosomes that belong to a chimaera with a degree of chimerism less than about 50%.

In some embodiments of the present application, under the condition that a plurality of embryos to be implanted from a same patient are provided, selecting and implantation priorities for the embryos are as follows:
the embryo with the genome-wide DNA methylation level within an interval of about 0.25 to 0.27 being in a first implantation priority;
the embryo with the genome-wide DNA methylation level within an interval of about 0.22 to 0.24 being in a second implantation priority;
the embryo with the genome-wide DNA methylation level within an interval of about 0.28 to 0.29 being in a third implantation priority;
the embryo with the genome-wide DNA methylation level within an interval of about 0.19 to 0.21 being in a fourth implantation priority; and
the embryo with the genome-wide DNA methylation level within an interval of about 0.30 to 0.33 being in a fifth implantation priority.

In some embodiments of the present application, under the condition that a plurality of embryos in a same implantation priority are provided, an embryo is selected as an implantation embryo from the plurality of embryos in the following preferential selecting priority:
the embryo with the lowest absolute difference value between the genome-wide DNA methylation level and 0.26 is preferentially implanted.

In some embodiments of the present application, testing the chromosomes includes pre-implantation genetic screening.

In some embodiments of the present application, the method further includes screening the embryos according to the methylation level of an imprinted gene control region or/and a gene promoter region to reduce a proportion of birth defects.

In some embodiments of the present application, testing the DNA methylation level includes constructing a methylation library and sequencing.

Details of one or more embodiments of the present application are set forth in the following description. Other features, objects, and advantages of the present application will become apparent from the description and claims of the present application.

### BRIEF DESCRIPTION OF THE DRAWINGS

The below will briefly describe drawings to be used in the description of embodiments of the present application to more clearly illustrate the technical solutions in embodiments of the present application, and to more completely understand the present application and its beneficial effects. Obviously, the drawings in the following description are only some embodiments of the present application and other drawings can also be obtained by those skilled in the art based on these drawings without creative work.

FIG. 1 is a flowchart of library construction in an embodiment of the present application.

FIG. 2 is a flowchart of data analysis in an embodiment of the present application.

FIG. 3 shows the pregnancy rate at different methylation levels.

FIG. 4 shows the abortion rate at different methylation levels.

FIG. 5 shows an embryo with deletion of chromosome 21.

FIG. 6 shows an embryo with deletion of a segment of chromosome 11.

FIG. 7 shows a comparison diagram of methylation levels of EMST gene promoter region in healthy-born embryos and abnormally developed embryos.

FIG. 8 shows a comparison diagram of methylation levels of CELSR2 gene promoter region in healthy-born embryos and abnormally developed embryos.

### DETAILED DESCRIPTION

Hereinafter, the technical solutions in embodiments of the present application will be described clearly and completely with reference to the drawings in the embodiments of the present application. Apparently, the described embodiments are only some of the embodiments of the present application, but not all of them. Other embodiments obtained by those of ordinary skill in the art without making creative efforts based on the embodiments of the present application all belong to the scope of protection of the present application.

Unless otherwise defined, all technical and scientific terms used herein have the same meaning as commonly understood by those skilled in the technical field to which this present disclosure belongs. The terms used herein in the specification of the present disclosure are only for the purpose of describing specific embodiments and are not intended to limit the present disclosure.

### Term

Terms and phrases used herein have the following meanings unless otherwise stated or contradictory.

As used herein, the terms “and/or”, “or/and”, “as well as/or” mean that their selection ranges include any one of two or more of the associated listed items, and any or all combinations of the associated listed items. The any or all combinations include the combination of any two of the associated listed items, the combination of any more of the associated listed items, or the combination of all associated listed items. It should be noted that when at least three items are connected by at least two conjunction combinations selected from “and/or”, “or/and” and “as well as/or”, it is understood that the technical embodiment in the present application undoubtedly includes both technical solutions that are all connected by “logic and”, and technical solutions that are all connected by “logic or”. In an example, “A and/or B” includes three parallel solutions of A, B, and A+B. In another example, the technical embodiment of “A, and/or, B, and/or, C, and/or, D” includes any one of A, B, C and D (i.e., the technical solutions that are all connected by “logic or”), and any or all combinations of A, B, C, and D, that is, including both combinations of any two or any three of A, B, C, and D, and the combination of A, B, C, and D (i.e., the technical solutions that are all connected by “logic and”).

Unless otherwise specified, “a plurality of”, “multiple”, “several”, etc., referred to in the present application mean that the number is greater than or equal to two. For example, “one or more” means one, or equal to or greater than two.

As used herein, “combinations thereof”, “any combination thereof”, “any combination way thereof”, etc., include all suitable combinations of any two or any more than two items of the listed items.

Herein, “suitable” in “suitable combination way”, “suitable way”, “any suitable way”, etc., be subject to being capable of implementing the technical solutions of the present application, solving the technical problem of the present application, and realizing the expected technical effects of the present application.

Herein, “preferable”, “better”, “superior”, “appropriate” are merely for describing embodiments or examples having good effects. It is understood that they do not constitute a limitation to the protection scope of the present application.

In the present application, “further”, “furthermore”, “particularly”, etc., are used for description purposes, indicating differences in content, but should not be construed as limiting the protection scope of the present application.

In the present application, “optionally”, “optional” mean that it may be present or may be absent, that is any one selected from two parallel solutions of “presence” or “absence”. If multiple repeats of “optionally” are present in one technical solution, each “optional” is independent when there is no contradiction or mutual restriction relationship, unless otherwise specified.

In the present application, the technical features described in open type include both closed-type technical solutions consisting of the listed features, and open-type technical solutions including the listed features.

In the present application, unless otherwise specified, when numerical intervals (i.e., numerical ranges) are referred to, the optional numerical distribution is considered continuous within the above numerical intervals, and includes both numerical endpoints of the numerical range (i.e., the minimum value and the maximum value), and every numeric value between the both numeric endpoints. Unless otherwise specified, the numerical interval when referring to the integers only within the numerical interval, includes the integers at both endpoints of the numerical range, and every integer between both endpoints. Herein, it is equivalent to directly enumerating each integer. For example, t being an integer selected from 1 to 10, means that t is any integer selected from the integer group consisting of 1, 2, 3, 4, 5, 6, 7, 8, 9 and 10. Furthermore, when multiple ranges are provided to describe a feature or a characteristic, these ranges may be combined. In other words, ranges disclosed herein are to be construed to include any or all sub-ranges subsumed therein, unless otherwise indicated.

In the present application, a temperature parameter is allowed to refer to not only a constant temperature treatment, but also a variation within a certain temperature range. It is understood that the described constant temperature treatment allows the temperature to fluctuate within the precision range under the control of an instrument. For example, the fluctuation within the range of ±5° C., ±4° C., t3° C., ±2° C., or ±1° C. is allowed.

In the present application, both % (w/w) and wt % represent percentage by weight, % (v/v) represents percentage by volume, and % (w/v) represents percentage by mass to volume.

All documents mentioned in the present application are incorporated herein by reference as if each document is individually cited as a reference. Unless conflicting with the objects and/or technical solutions of the present application, the cited documents involved in this application are incorporated in their entireties for all purposes. When cited documents are involved in this application, the definitions of relevant technical features, terms, nouns, phrases, etc. in the cited documents are also cited. When cited documents are involved in this application, the examples and preferred modes of the cited related technical features can also be incorporated herein as a reference, but only to the extent that it is possible to carry out the present application. It is understood that when the cited content conflicts with the description of the present application, the present application shall prevail or the cited content is amended adaptively according to the description of the present application.

After the embryo develops into a morula, the morula further divides, and the liquid secreted from the uterine cavity penetrates into the morula through the zona pellucida to gradually create a cystic cavity, called a blastocoel. The trophoblast cells refer to a layer of flat cells with a small size surrounding the outside of the blastocoel and will develop into a part of the placenta.

Embodiments of the present application provide a method for screening pre-implantation embryos, the method including the following steps:
testing chromosomes and DNA methylation levels of the pre-implantation embryos, and
selecting, from the pre-implantation embryos according to the obtained test result, the embryo having normal chromosomes, and a genome-wide DNA methylation level within an interval of about 0.25 to 0.27 or a genome-wide DNA methylation level outside the interval of about 0.25 to 0.27 but close to the interval of about 0.25 to 0.27 relative to other embryos as the embryo to be implanted.

In some embodiments of the present application, the pre-implantation embryos are embryos that have developed in vitro for about 5 to 7 days after fertilization.

In some embodiments of the present application, the pre-implantation embryos are embryos at a blastocyst stage.

In some embodiments of the present application, the sample to be tested includes a trophoblast cell from the pre-implantation embryo.

It should be noted that the optimal methylation level will vary depending on the blastocysts with different development days. Any strategy for recommending the implantation priorities based on the DNA methylation level of blastocysts is within the scope of protection of the present application.

In some embodiments of the present application, the normal chromosomes include chromosomes with a normal number and a normal structure, or chromosomes that belong to a chimaera with a degree of chimerism less than 50%.

In the screening method provided in the present application, if an embryo has a genome-wide DNA methylation level within an interval of about 0.25 to 0.27 among the pre-implantation embryos for screening, the embryo is preferentially implanted. It can be understood that the embryo having the genome-wide DNA methylation level within the interval of about 0.25 to 0.27 may not exist among the pre-implantation embryos for screening. In this case, the embryo having the genome-wide DNA methylation level relatively close to the interval is selected for implantation. As the genome-wide DNA methylation level is closer to the interval, the effect of assisted reproduction is better.

In some embodiments of the present application, under the condition that a plurality of embryos to be implanted from a same patient are provided, selecting and implantation priorities for the embryos are as follows:
the embryo with the genome-wide DNA methylation level within an interval of about 0.25 to 0.27 being in a first implantation priority;
the embryo with the genome-wide DNA methylation level within an interval of about 0.22 to 0.24 being in a second implantation priority;
the embryo with the genome-wide DNA methylation level within an interval of about 0.28 to 0.29 being in a third implantation priority;
the embryo with the genome-wide DNA methylation level within an interval of about 0.19 to 0.21 being in a fourth implantation priority; and
the embryo with the genome-wide DNA methylation level within an interval of about 0.30 to 0.33 being in a fifth implantation priority.

In some embodiments of the present application, under the condition that a plurality of embryos in a same implantation priority are provided, an embryo is selected as an implantation embryo from the plurality of embryos in the following preferential selecting and implantation priority:
an embryo is selected as an implantation embryo from the plurality of embryos in the following preferential selecting priority;
the embryo with the lowest absolute difference value between the genome-wide DNA methylation level and 0.26 is preferentially implanted.

The test results of a plurality of pre-implantation embryos from different families, namely having different fathers and different mothers, show that when the embryos are implanted according to the implantation priorities designated according to the above intervals, the corresponding birth rate increases sequentially. This also applies to a plurality of pre-implantation embryos from a same family, namely having the same father and the same mother. For example, based on the same family, if two embryos to be implanted are selected, one embryo with the genome-wide DNA methylation level of about 0.24 and the other embryo with the DNA methylation level of about 0.28, the former is preferentially selected.

During implantation of embryos, when the average genome-wide DNA methylation level of the embryo is close to 0.26, birth failure or birth defect may still occur after implantation of the embryo. This situation is mainly due to erroneous methylation levels of certain key regions on the genome. There are about 30 million CpG sites that can undergo methylation modifications on the human genome. The overall methylation level may not be affected when some of the sites are methylated incorrectly. However, if the changes in the methylation modification of those sites exactly affect the expression of a key gene, abortion after the implantation of the embryo or birth defect in a fetus may be caused.

Therefore, during the implementation of the present application, the methylation level of a gene promoter region or an imprinted gene control region is also considered to screen for epigenetic diseases.

In some embodiments of the present application, the screening method further includes the following step:
screening the embryos according to the methylation level of an imprinted gene control region or/and a gene promoter region to reduce a proportion of birth defects.

The target region described in the present application includes the known imprinted gene control region and gene promoter region on the human genome. The imprinted gene control regions in Table 2 and the gene promoter regions in Table 3 are only used to illustrate the implementation of the present application, but do not limit the scope of the present application. Any method for screening embryos through the DNA methylation level of the imprinted gene control region and the DNA methylation level of the promoter region is within the protection scope of the present application. It should be understood that the methods for screening embryos through the DNA methylation level of a single or a plurality of imprinted gene control regions alone, or the DNA methylation level of a single or a plurality of promoter regions alone, or the combination of the DNA methylation level of the single or the plurality of imprinted gene control regions and the DNA methylation level of the single or the plurality of promoter regions are all within the protection scope of the present application. The methylation levels of some imprinted gene control regions in abnormally developed embryos are shown in Table 1.

TABLE 1
Methylation levels of some imprinted gene control regions in
abnormally developed embryos

| Chr | Start | End | Gene | Rate |
|---|---|---|---|---|
| chr1 | 7887086 | 7888172 | M_PER3 | 20.00% |
| chr1 | 68512352 | 68513482 | M_DIRAS3 | 7.14% |
| chr1 | 68515436 | 68516492 | M_DIRAS3 | 8.33% |
| chr1 | 68517226 | 68517890 | M_DIRAS3 | 41.38% |
| chr3 | 182815724 | 182817626 | M_MCCC1 | 52.69% |
| chr6 | 3848242 | 3851373 | M_FAM50B | 31.98% |
| chr6 | 106956998 | 106958894 | M_CRYBG1 | 53.49% |
| chr7 | 50848952 | 50851051 | M_GRB10 | 65,03% |
| chr7 | 94285684 | 94287773 | M_TFPI2 | 21.00% |
| chr7 | 94953113 | 94953970 | M_PDK4 | 25.81% |
| chr7 | 130130219 | 130133591 | M_MEST | 38.39% |
| chr7 | 134671023 | 134671943 | M_AGBL3 | 11.43% |
| chr9 | 4297328 | 4300305 | M_GLIS3 | 23.63% |
| chr10 | 121577454 | 121578625 | M_INPP5F | 23.08% |
| chr11 | 2019263 | 2025009 | P_H19 | 20.92% |
| chr11 | 109962092 | 109965047 | M_ZC3H12C | 9.57% |
| chr13 | 33000310 | 33002993 | M_N4BP2L1 | 17.42% |
| chr13 | 48890306 | 48895970 | M_RB1 | 11.45% |
| chr14 | 101275427 | 101278058 | P_DLK1 | 1.56% |
| chr15 | 25199712 | 25201975 | M_PWRN1 | 12.17% |
| chr16 | 3481449 | 3482311 | M_ZNF597 | 69.44% |
| chr19 | 10304183 | 10306236 | M_DNMT1 | 53.10% |
| chr19 | 54039870 | 54042514 | M_ZNF331 | 4.61% |
| chr19 | 54150514 | 54154177 | M_MIR512-1 | 46.45% |
| chr19 | 57348071 | 57352657 | M_ZNF542P | 3.20% |
| chr20 | 36147193 | 36150305 | M_BLCAP | 29.94% |
| chr20 | 57424981 | 57431283 | M_MIR296 | 9.27% |
| chr21 | 48081256 | 48083366 | M_PRMT2 | 38.84% |
| chr22 | 42077737 | 42079313 | M_SNU13 | 36.84% |

TABLE 2
Imprinted gene control regions

| chr | ICR_start | ICR_end | gene | Methylation_allele |
|---|---|---|---|---|
| chr1 | 6684536 | 6686860 | THAP3 | P |
| chr1 | 7887086 | 7888172 | UTS2 | P |
| chr1 | 7887086 | 7888172 | PER3 | M |
| chr1 | 36183281 | 36185539 | AGO1 | P |
| chr1 | 55504789 | 55506760 | ACOT11 | P |
| chr1 | 59741536 | 59742152 | CYP2J2 | P |
| chr1 | 67772870 | 67774440 | IL12RB2 | P |
| chr1 | 68512352 | 68513482 | DIRAS3 | P |
| chr1 | 68515436 | 68516492 | DIRAS3 | P |
| chr1 | 68517226 | 68517890 | DIRAS3 | P |
| chr1 | 181556744 | 181557863 | CACNA1E | P |
| chr1 | 211589658 | 211591011 | LINC00467 | P |
| chr10 | 11934670 | 11937778 | PROSER2 | P |
| chr10 | 11934670 | 11937778 | PROSER2-AS1 | P |
| chr10 | 15760353 | 15762483 | ITGA8 | P |
| chr10 | 27702137 | 27703444 | PTCHD3 | P |
| chr10 | 65800981 | 65802070 | JMJD1C | P |
| chr10 | 71891899 | 71893159 | AIFM2 | P |
| chr10 | 104471289 | 104471461 | USMG5 | P |
| chr10 | 115998880 | 116000082 | VWA2 | P |
| chr10 | 121577454 | 121578625 | INPP5F | P |
| chr10 | 125649957 | 125652421 | CPXM2 | P |
| chr11 | 626306 | 628677 | IRF7 | P |
| chr11 | 2019263 | 2025009 | H19 | P |
| chr11 | 2019263 | 2025009 | MIR675 | P |
| chr11 | 2019263 | 2025009 | IGF2 | P |
| chr11 | 2019263 | 2025009 | MIR483 | P |
| chr11 | 2019263 | 2025009 | IGF2-AS | P |
| chr11 | 2019263 | 2025009 | INS | P |
| chr11 | 2719780 | 2722440 | KCNQ1 | P |
| chr11 | 2719780 | 2722440 | KCNQ1OT1 | P |
| chr11 | 2719780 | 2722440 | KCNQ1DN | P |
| chr11 | 2719780 | 2722440 | CDKN1C | P |
| chr11 | 2719780 | 2722440 | SLC22A18 | P |
| chr11 | 2719780 | 2722440 | PHLDA2 | P |
| chr11 | 3573847 | 3574161 | ART5 | P |
| chr11 | 7098198 | 7098782 | ZNF215 | P |
| chr11 | 7693999 | 7695837 | OVCH2 | P |
| chr11 | 19366234 | 19368233 | NAV2 | P |
| chr11 | 44087366 | 44089013 | ALKBH3 | P |
| chr11 | 44087366 | 44089013 | ACCS | P |
| chr11 | 45201576 | 45203519 | MAPK81P1 | P |
| chr11 | 77533979 | 77534239 | PAK1 | P |
| chr11 | 93582790 | 93584557 | VSTM5 | P |
| chr11 | 109962092 | 109965047 | ZC3H12C | P |
| chr11 | 131451773 | 131452206 | NTM | P |
| chr11 | 131707563 | 131708141 | NTM | P |
| chr1] | 131995788 | 131997591 | NTM | P |
| chr11 | 132812314 | 132814852 | OPCML | P |
| chr11 | 132933757 | 132935979 | OPCML | P |
| chr11 | 132950967 | 132954262 | OPCML | P |
| chr12 | 2338398 | 2339942 | CACNA1C | P |
| chr12 | 2799932 | 2801629 | CACNA1C | P |
| chr12 | 4029805 | 4030544 | TIGAR | P |
| chr12 | 65514878 | 65516418 | WIF1 | P |
| chr13 | 33000310 | 33002993 | N4BP2L1 | P |
| chr13 | 48890306 | 48895970 | RB1 | P |
| chr13 | 48890306 | 48895970 | LPAR6 | P |
| chr13 | 70681275 | 70682652 | KLHL1 | P |
| chr13 | 102568071 | 102570612 | FGF14 | P |
| chr14 | 24069635 | 24070253 | PCK2 | P |
| chr14 | 101275427 | 101278058 | DLK1 | P |
| chr14 | 101275427 | 101278058 | MEG3 | P |
| chr14 | 101275427 | 101278058 | MIR337 | P |
| chr14 | 101275427 | 101278058 | RTL1 | P |
| chr14 | 101275427 | 101278058 | MEG8 | P |
| chr14 | 101275427 | 101278058 | MIR134 | P |
| chr15 | 25199712 | 25201975 | PWRN1 | P |
| chr15 | 25199712 | 25201975 | NPAP1 | P |
| chr15 | 25199712 | 25201975 | SNRPN | P |
| chr15 | 25199712 | 25201975 | SNHG14 | P |
| chr15 | 25199712 | 25201975 | SNURF | P |
| chr15 | 25199712 | 25201975 | SNORD107 | P |
| chr15 | 25199712 | 25201975 | SNORD64 | P |
| chr15 | 25199712 | 25201975 | SNORD108 | P |
| chr15 | 25199712 | 25201975 | SNORD109B | P |
| chr15 | 25199712 | 25201975 | IPW | P |
| chr15 | 25199712 | 25201975 | UBE3A | P |
| chr15 | 33602047 | 33604174 | RYR3 | P |
| chr15 | 75412344 | 75413396 | DNM1P35 | P |
| chr15 | 79327672 | 79328768 | RASGRF1 | P |
| chr15 | 93197772 | 93199531 | FAM174B | P |
| chr15 | 99122903 | 99124148 | IRAIN | P |
| chr15 | 102479168 | 102479705 | LRRK1 | P |
| chr16 | 3481449 | 3482311 | ZNF597 | P |
| chr16 | 3481449 | 3482311 | NAA60 | P |
| chr16 | 48398663 | 48400563 | SIAH1 | P |
| chr16 | 53543981 | 53544823 | RBL2 | P |
| chr16 | 68572341 | 68574155 | ZFP90 | P |
| chr16 | 78804359 | 78804965 | CLEC3A | P |
| chr17 | 7643548 | 7644419 | NLGN2 | P |
| chr19 | 3403323 | 3405383 | GNG7 | P |
| chr19 | 10304183 | 10306236 | DNMT1 | P |
| chr19 | 10304183 | 10306236 | S1PR2 | P |
| chr19 | 10304183 | 10306236 | ICAM1 | P |
| chr19 | 10304183 | 10306236 | FDX1L | P |
| chr19 | 11783946 | 11786066 | ZNF833P | P |
| chr19 | 13614721 | 13618358 | CACNA1A | P |
| chr19 | 17345621 | 17346885 | ANO8 | P |
| chr19 | 21097472 | 21097767 | ZNF714 | P |
| chr19 | 54039870 | 54042514 | ZNF331 | P |
| chr19 | 54150514 | 54154177 | MIR512-1 | P |
| chr19 | 57348071 | 57352657 | ZNF542P | P |
| chr19 | 57348071 | 57352657 | ZIM2 | P |
| chr19 | 57348071 | 57352657 | PEG3 | P |
| chr19 | 57348071 | 57352657 | MIMT1 | P |
| chr2 | 40299700 | 40300150 | THUMPD2 | P |
| chr2 | 46655893 | 46657036 | TMEM247 | P |
| chr2 | 80816191 | 80816303 | LRRTM1 | P |
| chr2 | 114298625 | 114300145 | PAX8 | P |
| chr2 | 114298625 | 114300145 | PAX8-AS1 | P |
| chr2 | 203638751 | 203639365 | ICA1L | P |
| chr2 | 206789262 | 206791183 | GPR1 | P |
| chr2 | 206789262 | 206791183 | GPR1-AS | P |
| chr2 | 207521578 | 207521737 | ZDBF2 | P |
| chr2 | 224895869 | 224896575 | MRPL44 | P |
| chr2 | 229044929 | 229047110 | SPHKAP | P |
| chr20 | 23520000 | 23520386 | CST1 | P |
| chr20 | 36147193 | 36150305 | BLCAP | P |
| chr20 | 36147193 | 36150305 | NNAT | P |
| chr20 | 42142256 | 42144161 | L3MBTL1 | P |
| chr20 | 42142256 | 42144161 | SGK2 | P |
| chr20 | 42142256 | 42144161 | GDAP1L1 | P |
| chr20 | 52789044 | 52791521 | CYP24A1 | P |
| chr20 | 57424981 | 57431283 | MIR296 | P |
| chr20 | 57424981 | 57431283 | MIR298 | P |
| chr20 | 57424981 | 57431283 | GNAS-AS1 | P |
| chr20 | 57424981 | 57431283 | GNAS | P |
| chr20 | 57424981 | 57431283 | GNAS | P |
| chr20 | 57424981 | 57431283 | GNAS | P |
| chr21 | 37476783 | 37477201 | CBR1 | P |
| chr21 | 48081256 | 48083366 | PRMT2 | P |
| chr22 | 17089583 | 17091485 | TPTEP1 | P |
| chr22 | 40057761 | 40061173 | CACNA1I | P |
| chr22 | 42077737 | 42079313 | SNU13 | P |
| chr3 | 21447252 | 21448120 | ZNF385D | P |
| chr3 | 27503852 | 27506672 | SLC4A7 | P |
| chr3 | 49312204 | 49315853 | USP4 | P |
| chr3 | 128335974 | 128337510 | RAB7A | P |
| chr3 | 128335974 | 128337510 | EFCC1 | P |
| chr3 | 182815724 | 182817626 | MCCC1 | P |
| chr3 | 192444749 | 192445594 | FGF12 | P |
| chr3 | 196728469 | 196731392 | MELTF | P |
| chr4 | 4038135 | 4039176 | STX18-AS1 | P |
| chr4 | 9019892 | 9020175 | GPR78 | P |
| chr4 | 89618382 | 89619639 | NAP1L5 | P |
| chr4 | 93224894 | 93227298 | GRID2 | P |
| chr4 | 187071083 | 187071852 | FAM149A | P |
| chr4 | 190783839 | 190784136 | FRG1 | P |
| chr5 | 95066149 | 95068034 | RHOBTB3 | P |
| chr5 | 102897486 | 102899693 | NUDT12 | P |
| chr5 | 135114070 | 135114210 | VTRNA2-1 | P |
| chr5 | 178593500 | 178594990 | ZNF354C | P |
| chr6 | 3848242 | 3851373 | FAM50B | P |
| chr6 | 14117422 | 14118919 | CD83 | P |
| chr6 | 18368625 | 18369036 | RNF144B | P |
| chr6 | 37615794 | 37618741 | MDGA1 | P |
| chr6 | 39901465 | 39902599 | MOCS1 | P |
| chr6 | 105401218 | 105401823 | LIN28B | P |
| chr6 | 106956998 | 106958894 | CRYBG1 | P |
| chr6 | 144328299 | 144330016 | PHACTR2 | P |
| chr6 | 144328299 | 144330016 | PLAGL1 | P |
| chr6 | 144328299 | 144330016 | HYMAI | P |
| chr6 | 160715121 | 160715655 | SLC22A2 | P |
| chr6 | 160720644 | 160721781 | SLC22A3 | P |
| chr6 | 161022727 | 161023102 | PLG | P |
| chr6 | 169100205 | 169100724 | KIF25 | P |
| chr7 | 10929327 | 10929756 | THSD7A | P |
| chr7 | 12609659 | 12611226 | SCIN | P |
| chr7 | 22765719 | 22767571 | RAPGEF5 | P |
| chr7 | 50848952 | 50851051 | GRB10 | P |
| chr7 | 92672381 | 92673113 | CALCR | P |
| chr7 | 94285684 | 94287773 | TFPI2 | P |
| chr7 | 94285684 | 94287773 | SGCE | P |
| chr7 | 94285684 | 94287773 | PEG10 | P |
| chr7 | 94953113 | 94953970 | PDK4 | P |
| chr7 | 129747420 | 129749246 | KLHDC10 | P |
| chr7 | 129910979 | 129913008 | CPA4 | P |
| chr7 | 130130219 | 130133591 | MEST | P |
| chr7 | 130130219 | 130133591 | MEST1T1 | P |
| chr7 | 130130219 | 130133591 | KLF14 | P |
| chr7 | 134671023 | 134671943 | AGBL3 | P |
| chr7 | 151298633 | 151299251 | PRKAG2 | P |
| chr8 | 831978 | 833586 | DLGAP2 | P |
| chr8 | 1320857 | 1322838 | DLGAP2 | P |
| chr8 | 8558896 | 8560866 | CLDN23 | P |
| chr8 | 80483569 | 80484764 | PKIA | P |
| chr8 | 135937671 | 135939330 | ZFAT | P |
| chr8 | 135937671 | 135939330 | ZFAT-AS1 | P |
| chr8 | 140642125 | 140643756 | KCNK9 | P |
| chr8 | 141106930 | 141111080 | TRAPPC9 | P |
| chr8 | 141106930 | 141111080 | PEG13 | P |
| chr8 | 142939147 | 142939701 | DENND3 | P |
| chr8 | 143130636 | 143131150 | PSCA | P |
| chr9 | 72698 | 74301 | PGM5P3-AS1 | P |
| chr9 | 4297328 | 4300305 | GLIS3 | P |
| chr9 | 140300869 | 140303228 | EXD3 | P |
| chr11 | 12041235 | 12041270 | RNF141 | P |
| chr11 | 30022087 | 30022507 | WT1 | P |
| chr11 | 30022087 | 30022507 | WT1-AS | P |
| chr11 | 35843093 | 35843399 | LINC00294 | P |
| chr11 | 71382582 | 71383262 | ANO1 | P |
| chr11 | 125743712 | 125744319 | SPA17 | P |
| chr13 | 50231393 | 50232219 | DLEU7 | P |
| chr15 | 25199712 | 25201975 | MKRN3 | P |
| chr15 | 25199712 | 25201975 | MAGEL2 | P |
| chr15 | 25199712 | 25201975 | NDN | P |
| chr16 | 68572341 | 68574155 | PDPR | P |
| chr17 | 52908852 | 52909780 | SEPT4 | P |
| chr19 | 43045944 | 43046839 | AXL | P |
| chr2 | 195202912 | 195203181 | DNAH7 | P |
| chr20 | 33762054 | 33764608 | ACTL10 | P |
| chr20 | 42142256 | 42144161 | ZHX3 | P |
| chr22 | 17089583 | 17091485 | ARVCF | P |
| chr4 | 3970877 | 3970994 | PDE6B | P |
| chr4 | 3970877 | 3970994 | SH3BP2 | P |
| chr5 | 81763802 | 81764160 | RNU5D-1 | P |
| chr6 | 28151953 | 28152215 | C6orf47 | P |
| chr6 | 44528720 | 44529409 | CUL7 | P |
| chr8 | 22033741 | 22035013 | R3HCC1 | P |
| chr8 | 25911037 | 25911407 | PTK2B | P |
| chr8 | 145954428 | 145956024 | NAPRT | P |
| chrX | 48712407 | 48713431 | SLC9A7 | P |
| chr17 | 71087887 | 71090143 | SLC39A11 | P |
| chr2 | 230802843 | 230805064 | FBXO36 | P |
| chr7 | 22765719 | 22767571 | IL6 | P |
| chr15 | 64673065 | 64674447 | PCLAF | P |
| chr6 | 122803205 | 122803989 | PKIB | P |
| chr21 | 44346819 | 44347847 | ERVH48-1 | P |
| chr11 | 68472586 | 68473682 | GAL | P |
| chr14 | 47099332 | 47099819 | RPL10L | P |
| chr15 | 25199712 | 25201975 | PWARSN | P |
| chr10 | 135383339 | 135383585 | CYP2E1 | P |
| chr5 | 133817520 | 133820270 | LOC102546229 | P |
| chr18 | 77658265 | 77660315 | HSBP1L1 | P |
| chr7 | 97382013 | 97382335 | ASNS | P |
| chr21 | 20041410 | 20041958 | MIR548XHG | M |
| chr4 | 124694116 | 124694593 | LINC01091 | M |
| chr10 | 135383339 | 135383585 | SYCE1 | M |
| chr6 | 27941532 | 27941923 | HIST1H2AM | M |
| chr15 | 55452723 | 55452936 | PIGB | M |
| chr12 | 8474160 | 8474785 | CLEC4D | M |

TABLE 3
Gene promoter regions

| | | | |
|---|---|---|---|
| chr1 | 6684426 | 6685426 | THAP3 |
| chr1 | 7843880 | 7844880 | PER3 |
| chr1 | 7913072 | 7914072 | UTS2 |
| chr1 | 36334909 | 36335909 | AGO1 |
| chr1 | 45196342 | 45197342 | RNU5D-1 |
| chr1 | 55007430 | 55008430 | ACOT11 |
| chr1 | 60391962 | 60392962 | CYP2J2 |
| chr1 | 67772547 | 67773547 | IL12RB2 |
| chr1 | 68516814 | 68517814 | DIRAS3 |
| chr1 | 159821262 | 159822262 | SNORD64 |
| chr1 | 181381738 | 181382738 | CACNA1E |
| chr1 | 211555645 | 211556645 | LINC00467 |
| chr2 | 40005907 | 40006907 | THUMPD2 |
| chr2 | 46655829 | 46656829 | TMEM247 |
| chr2 | 80531374 | 80532374 | LRRTM1 |
| chr2 | 113968599 | 113969599 | PAX8-AS1 |
| chr2 | 114036027 | 114037027 | PAX8 |
| chr2 | 196933036 | 196934036 | DNAH7 |
| chr2 | 203736208 | 203737208 | ICA1L |
| chr2 | 207082271 | 207083271 | GPR1 |
| chr2 | 207138887 | 207139887 | ZDBF2 |
| chr2 | 224821621 | 224822621 | MRPL44 |
| chr2 | 229045861 | 229046861 | SPHKAP |
| chr2 | 230786518 | 230787518 | FBXO36 |
| chr3 | 22414312 | 22415312 | ZNF385D |
| chr3 | 27525411 | 27526411 | SLC4A7 |
| chr3 | 49377645 | 49378645 | USP4 |
| chr3 | 128444465 | 128445465 | RAB7A |
| chr3 | 128719972 | 128720972 | EFCC1 |
| chr3 | 182833363 | 182834363 | MCCC1 |
| chr3 | 192485053 | 192486053 | FGF12 |
| chr4 | 618873 | 619873 | PDE6B |
| chr4 | 2794250 | 2795250 | SH3BP2 |
| chr4 | 4543358 | 4544358 | STX18-AS1 |
| chr4 | 8559952 | 8560952 | GPR78 |
| chr4 | 89618886 | 89619886 | NAP1L5 |
| chr4 | 93225050 | 93226050 | GRID2 |
| chr4 | 124570922 | 124571922 | LINC01091 |
| chr4 | 187025073 | 187026073 | FAM149A |
| chr4 | 190861443 | 190862443 | FRG1 |
| chr5 | 95048726 | 95049726 | RHOBTB3 |
| chr5 | 102897994 | 102898994 | NUDT12 |
| chr5 | 135415786 | 135416786 | VTRNA2-1 |
| chr5 | 178486916 | 178487916 | ZNF354C |
| chr6 | 3849120 | 3850120 | FAM50B |
| chr6 | 14117372 | 14118372 | CD83 |
| chr6 | 18368279 | 18369279 | RNF144B |
| chr6 | 27860463 | 27861463 | HIST1H2AM |
| chr6 | 31628049 | 31629049 | C6orf47 |
| chr6 | 37666582 | 37667582 | MDGA1 |
| chr6 | 39901790 | 39902790 | MOCS1 |
| chr6 | 43021183 | 43022183 | CUL7 |
| chr6 | 105404423 | 105405423 | LIN28B |
| chr6 | 122792576 | 122793576 | PKIB |
| chr6 | 143857482 | 143858482 | PHACTR2 |
| chr6 | 144385235 | 144386235 | PLAGL1 |
| chr6 | 160698170 | 160699170 | SLC22A2 |
| chr6 | 160768800 | 160769800 | SLC22A3 |
| chr6 | 161122770 | 161123770 | PLG |
| chr6 | 168396421 | 168397421 | KIF25 |
| chr7 | 11871324 | 11872324 | THSD7A |
| chr7 | 12609703 | 12610703 | SCIN |
| chr7 | 22396263 | 22397263 | RAPGEF5 |
| chr7 | 22765003 | 22766003 | IL6 |
| chr7 | 50860659 | 50861659 | GRB10 |
| chr7 | 93203542 | 93204542 | CALCR |
| chr7 | 93519803 | 93520803 | TFPI2 |
| chr7 | 94285021 | 94286021 | SGCE |
| chr7 | 94285137 | 94286137 | PEG10 |
| chr7 | 95225303 | 95226303 | PDK4 |
| chr7 | 97501354 | 97502354 | ASNS |
| chr7 | 29709850 | 129710850 | KLHDC10 |
| chr7 | 129932474 | 129933474 | CPA4 |
| chr7 | 130125512 | 130126512 | MEST |
| chr7 | 130418388 | 130419388 | KLF14 |
| chr7 | 134670759 | 134671759 | AGBL3 |
| chr7 | 151573710 | 151574710 | PRKAG2 |
| chrX | 46617990 | 46618990 | SLC9A7 |
| chr8 | 1449032 | 1450032 | DLGAP2 |
| chr8 | 8558948 | 8559948 | CLDN23 |
| chr8 | 23127133 | 23128133 | R3HCC1 |
| chr8 | 27168499 | 27169499 | PTK2B |
| chr8 | 79427874 | 79428874 | PKIA |
| chr8 | 135724792 | 135725792 | ZFAT |
| chr8 | 135609814 | 135610814 | ZFAT-AS1 |
| chr8 | 140714799 | 140715799 | KCNK9 |
| chr8 | 141468178 | 141469178 | TRAPPC9 |
| chr8 | 142126877 | 142127877 | DENND3 |
| chr8 | 143751226 | 143752226 | PSCA |
| chr9 | 4347892 | 4348892 | GLIS3 |
| chr9 | 140317214 | 140318214 | EXD3 |
| chr10 | 11864838 | 11865838 | PROSER2 |
| chr10 | 11936199 | 11937199 | PROSER2-AS1 |
| chr10 | 15761624 | 15762624 | ITGA8 |
| chr10 | 27702797 | 27703797 | PTCHD3 |
| chr10 | 65225222 | 65226222 | JMJD1C |
| chr10 | 71892190 | 71893190 | AIFM2 |
| chr10 | 105155723 | 105156723 | USMG5 |
| chr10 | 115998518 | 115999518 | VWA2 |
| chr10 | 121485109 | 121486109 | INPP5F |
| chr10 | 125699283 | 125700283 | CPXM2 |
| chr10 | 135333410 | 135334410 | CYP2E1 |
| chr10 | 135382376 | 135383376 | SYCE1 |
| chr11 | 615499 | 616499 | IRF7 |
| chr11 | 2022200 | 2023200 | H19 |
| chr11 | 2170333 | 2171333 | IGF2 |
| chr11 | 2154939 | 2155939 | MIR483 |
| chr11 | 2161231 | 2162231 | IGF2-AS |
| chr11 | 2182071 | 2183071 | INS |
| chr11 | 2465414 | 2466414 | KCNQ1 |
| chr11 | 2720724 | 2721724 | KCNQ1OT1 |
| chr11 | 2890763 | 2891763 | KCNQ1DN |
| chr11 | 2906611 | 2907611 | CDKN1C |
| chr11 | 2920451 | 2921451 | SLC22A18 |
| chr11 | 2950185 | 2951185 | PHLDA2 |
| chr11 | 3663046 | 3664046 | ART5 |
| chr11 | 6947135 | 6948135 | ZNF215 |
| chr11 | 7727468 | 7728468 | OVCH2 |
| chr11 | 10562277 | 10563277 | RNF141 |
| chr11 | 19371771 | 19372771 | NAV2 |
| chr11 | 32456676 | 32457676 | WT1 |
| chr11 | 32456564 | 32457564 | WT1-AS |
| chr11 | 43901861 | 43902861 | ALKBH3 |
| chr11 | 44086975 | 44087975 | ACCS |
| chr11 | 45906702 | 45907702 | MAPK8IP1 |
| chr11 | 68450747 | 68451747 | GAL |
| chr11 | 69923908 | 69924908 | ANO1 |
| chr11 | 77185180 | 77186180 | PAK1 |
| chr11 | 93583197 | 93584197 | VSTM5 |
| chr11 | 109963587 | 109964587 | ZC3H12C |
| chr11 | 124543194 | 124544194 | SPA17 |
| chr11 | 131239873 | 131240873 | NTM |
| chr11 | 133401914 | 133402914 | OPCML |
| chr12 | 2079452 | 2080452 | CACNA1C |
| chr12 | 8661571 | 8662571 | CLEC4D |
| chr12 | 65514846 | 65515846 | WIF1 |
| chr13 | 33001815 | 33002815 | N4BP2L1 |
| chr13 | 48877387 | 48878387 | RB1 |
| chr13 | 49018340 | 49019340 | LPAR6 |
| chr13 | 51417575 | 51418575 | DLEU7 |
| chr13 | 70682091 | 70683091 | KLHL1 |
| chr13 | 103053624 | 103054624 | FGF14 |
| chr14 | 24562762 | 24563762 | PCK2 |
| chr14 | 47120528 | 47121528 | RPL10L |
| chr14 | 101191542 | 101192542 | DLK1 |
| chr14 | 101245247 | 101246247 | MEG3 |
| chr14 | 101340330 | 101341330 | MIR337 |
| chr14 | 101350684 | 101351684 | RTL1 |
| chr14 | 101360607 | 101361607 | MEG8 |
| chr14 | 101520508 | 101521508 | MIR134 |
| chr15 | 23809954 | 23810954 | MKRN3 |
| chr15 | 23890675 | 23891675 | MAGEL2 |
| chr15 | 23931950 | 23932950 | NDN |
| chr15 | 24737784 | 24738784 | PWRN1 |
| chr15 | 24920041 | 24921041 | NPAP1 |
| chr15 | 25068294 | 25069294 | SNRPN |
| chr15 | 25199633 | 25200633 | SNURF |
| chr15 | 25199681 | 25200681 | SNURF |
| chr15 | 25223230 | 25224230 | SNHG14 |
| chr15 | 25229511 | 25230511 | SNORD64 |
| chr15 | 25231572 | 25232572 | SNORD108 |
| chr15 | 25522990 | 25523990 | SNORD109B |
| chr15 | 25683628 | 25684628 | UBE3A |
| chr15 | 33602663 | 33603663 | RYR3 |
| chr15 | 55610658 | 55611658 | PIGB |
| chr15 | 76030464 | 76031464 | DNM1P35 |
| chr15 | 79382615 | 79383615 | RASGRF1 |
| chr15 | 93352614 | 93353614 | FAM174B |
| chr15 | 101458920 | 101459920 | LRRK1 |
| chr16 | 3414599 | 3415599 | NAA60 |
| chr16 | 3493042 | 3494042 | ZNF597 |
| chr16 | 3493111 | 3494111 | NAA60 |
| chr16 | 48481813 | 48482813 | SIAH1 |
| chr16 | 53467389 | 53468389 | RBL2 |
| chr16 | 68563493 | 68564493 | ZFP90 |
| chr16 | 70147029 | 70148029 | PDPR |
| chr16 | 78055912 | 78056912 | CLEC3A |
| chr17 | 7307693 | 7308693 | NLGN2 |
| chr17 | 56617679 | 56618679 | SEPT4 |
| chr17 | 71088351 | 71089351 | SLC39A11 |
| chr18 | 77724061 | 77725061 | HSBP1L1 |
| chr20 | 23731405 | 23732405 | CST1 |
| chr20 | 32253804 | 32254804 | ACTL10 |
| chr20 | 36155833 | 36156833 | BLCAP |
| chr20 | 36149117 | 36150117 | NNAT |
| chr20 | 39945812 | 39946812 | ZHX3 |
| chr20 | 42135820 | 42136820 | L3MBTL1 |
| chr20 | 42187108 | 42188108 | SGK2 |
| chr20 | 42875387 | 42876387 | GDAP1L1 |
| chr20 | 52790012 | 52791012 | CYP24A1 |
| chr20 | 57392280 | 57393280 | MIR296 |
| chr20 | 57392868 | 57393868 | MIR298 |
| chr20 | 57425458 | 57426458 | GNAS-AS1 |
| chr20 | 57414273 | 57415273 | GNAS |
| chr19 | 2702207 | 2703207 | GNG7 |
| chr19 | 10341462 | 10342462 | DNMT1 |
| chr19 | 10341448 | 10342448 | S1PR2 |
| chr19 | 10381011 | 10382011 | ICAM1 |
| chr19 | 10426191 | 10427191 | FDX1L |
| chr19 | 11750091 | 11751091 | ZNF833P |
| chr19 | 13734304 | 13735304 | CACNA1A |
| chr19 | 17445138 | 17446138 | ANO8 |
| chr19 | 21264465 | 21265465 | ZNF714 |
| chr19 | 41724608 | 41725608 | AXL |
| chr19 | 54023735 | 54024735 | ZNF331 |
| chr19 | 54169427 | 54170427 | MIR512-1 |
| chr19 | 57351597 | 57352597 | ZIM2 |
| chr19 | 57351596 | 57352596 | PEG3 |
| chr19 | 57351770 | 57352770 | MIMT1 |
| chr22 | 17082277 | 17083277 | TPTEP1 |
| chr22 | 20003831 | 20004831 | ARVCF |
| chr22 | 39966258 | 39967258 | CACNA1I |
| chr21 | 37441739 | 37442739 | CBR1 |
| chr21 | 44345256 | 44346256 | ERVH48-1 |
| chr21 | 48054579 | 48055579 | PRMT2 |

In some embodiments of the present application, testing the chromosomes includes preimplantation genetic screening.

In some embodiments of the present application, testing the DNA methylation level includes constructing a methylation library and sequencing.

### Specific Examples

Hereinafter, the embodiments of the present application will be described in detail in combination with examples. It is understood that these examples are only used to illustrate the present application and are not intended to limit the scope of the present application. The experimental methods, the specific conditions of which are not specified in the following examples, preferably refer to the guidelines given in the present application, and also can be performed according to the experimental manual or routine conditions in the art, or according to the conditions instructed by the manufacturer, or refer to the existing experimental methods known in the art.

In the following specific examples, the measurement parameters related to raw material components may have slight deviations within the weighing accuracy range, unless otherwise specified. The acceptable deviations are allowable for the involved temperature parameter and time parameter due to instrumental test accuracy or operational accuracy.

### 1. Obtaining Samples

A total of 180 volunteer families were recruited for the present application. Subjects signed the informed consent and were involved in the clinical trial groups after being approved by the ethics committee. After superovulation, oocyte retrieval, in vitro fertilization and embryo culture in vino, the trophoblast cells were biopsied.

### 2. Test of the Samples

### 2.1 Testing of DNA Methylation Level of Pre-Implantation Embryos

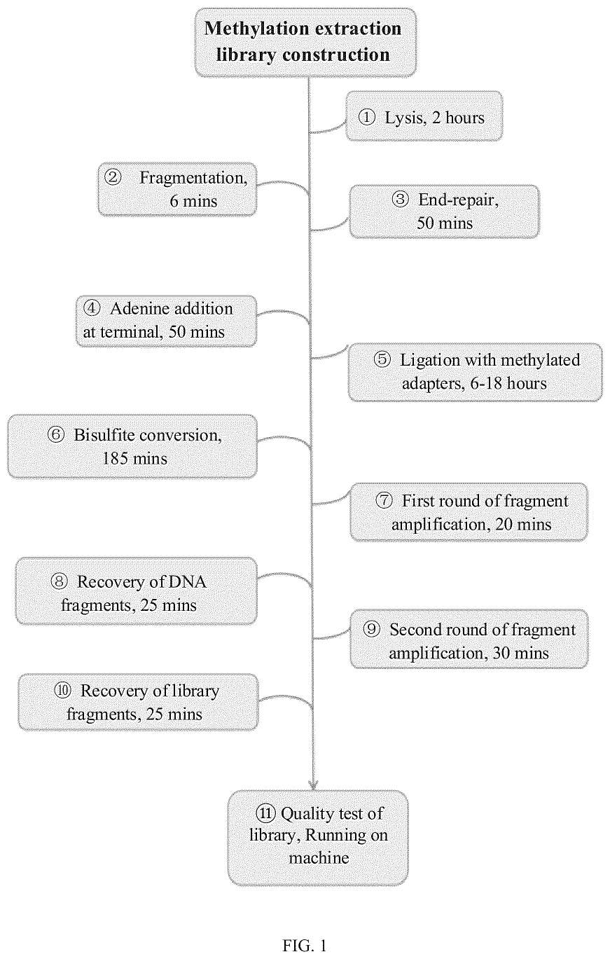

The obtained biopsy cells were subjected to genome-wide DNA methylation library construction. The flowchart of the library construction is shown in FIG. 1, including the following steps.

### 1. Lysis of Cells

A trophoblast cell sample to be tested was taken out from a refrigerator. A PCR tube containing the trophoblast cell sample was briefly centrifuged and placed on ice. A cell lysing solution was added to the PCR tube containing the trophoblast cell sample to lyse cells for two hours.

### 2. Fragmentation of DNA

After the cells were lysed, a buffer solution and a DNA protection solution were added. The DNA was broken into fragments of about 400 bp by using an ultrasonicator.

### 3. End-Repair

End-repair enzymes and end-repair buffer were added to the PCR tube containing the DNA fragments of the cell sample to be tested, mixed uniformly, and placed in a PCR machine for incubation. The PCR procedure was set as: 20° C. for 30 mins; 75° C. for 20 mins; and holding at 4° C.

### 4. Addition of Adenine at the Terminal

dATPs, DNA polymerases, ligation buffer and Tris-HCl solution were added to the end-repair product, mixed uniformly, and placed in the PCR machine for incubation. The PCR procedure was set as: 37° C. for 30 mins: 75° C. for 20 mins; and holding at 4° C.

### 5. Addition of an Adapter

Methylated adapters, ligases, ligation buffer and Tris-HCl solution were added to the product ligated with adenine at the terminal, mixed uniformly, and placed in a constant temperature cabinet for ligation at 16° C. for 12 hours.

### 6. Bisulfite Conversion

A CT conversion reagent was added to the product ligated with the adapter, mixed uniformly, and placed in the PCR machine to react for 2.5 hours.

The converted product was transferred to an adsorption column. A binding buffer was added, then mixed uniformly, left standing for 5 minutes, and the column was centrifuged for 1 minute. The liquid in a collection tube was discarded.

A washing solution was added to the adsorption column, then the column was centrifuged for 1 minute, and the liquid in the collection tube was discarded.

A desulfonation buffer was added to the adsorption column, and left standing for 15 minutes, then the column was centrifuged for 1 minute, and the liquid in the collection tube was discarded.

A washing solution was added to the adsorption column, then the column was centrifuged for 1 minute, and the liquid in the collection tube was discarded.

A washing solution was added to the adsorption column, then the column was centrifuged for 1 minute, and the liquid in the collection tube was discarded.

The adsorption column was transferred to a centrifuge tube, and the cover was kept open to dry at room temperature for 5 minutes.

A preheated eluent was added to the adsorption column and the column was centrifuged for 1 minute.

### 7. First Round of Amplification

The collected nucleic acid was transferred to a new PCR tube. Sequencing primers and PCR enzyme mixture 1 were added, and mixed uniformly, and the tube was placed in the PCR machine for amplification. The PCR procedure was set as: 98° C. for 45 seconds; 8 cycles of 98° C. for 15 seconds, 65° C. for 30 seconds, 72° C. for 30 seconds; 72° C. for 1 minute; and holding at 4° C.

### 8. Purification and Recovery

Magnetic beads were added to the amplified product, mixed uniformly, and left standing for 8 minutes.

The PCR tube was placed on a magnetic stand, left standing for 2 minutes, and the liquid was discarded.

Freshly prepared 80% ethanol was added to the PCR tube, left standing for 30 seconds, and the liquid was discarded.

Freshly prepared 80% ethanol was added to the PCR tube again, and left standing for 30 seconds, the liquid was discarded, and the cover was kept open to dry for 5 minutes.

An eluent was added to the PCR tube, mixed uniformly, and left standing for 5 minutes.

The PCR tube was placed on the magnetic stand, left standing for 2 minutes, and the liquid was transferred to a new PCR tube.

### 9. Second Round of Amplification

Universal primers and PCR enzyme mixed solution 2 were added to the PCR tube, mixed uniformly, and placed in the PCR machine for amplification. The PCR procedure was set as: 98° C. for 30 seconds; 9 to 12 cycles of 98° C. for 10 seconds, 65° C. for 75 seconds; 65° C. for 5 minutes; and holding at 4° C.

### 10. Purification and Recovery

Magnetic beads were added to the amplified product, mixed uniformly, and left standing for 8 minutes.

The PCR tube was placed on the magnetic stand, left standing for 2 minutes, and the liquid was discarded.

Freshly prepared 80% ethanol was added to the PCR tube, left standing for 30 seconds, and the liquid was discarded.

Freshly prepared aqueous ethanol solution with an ethanol content of 80% (v/v) was added to the PCR tube, left standing for 30 seconds, the liquid was discarded, and the cover was kept open to dry for 5 minutes.

Nuclease-free water was added to the PCR tube, mixed uniformly, and left standing for 5 minutes.

The PCR tube was placed on the magnetic stand, left standing for 2 minutes, and the liquid was transferred to a collection tube.

### 11. Quality Test of Library

The library was quantified using the Q-PCR instrument. Sizes of the library fragments were determined using an Agilent 2100.

### 12. Sequencing of Library

Once the quality of the library was satisfactory, the library was sequenced using the next generation sequencing (NGS) platform.

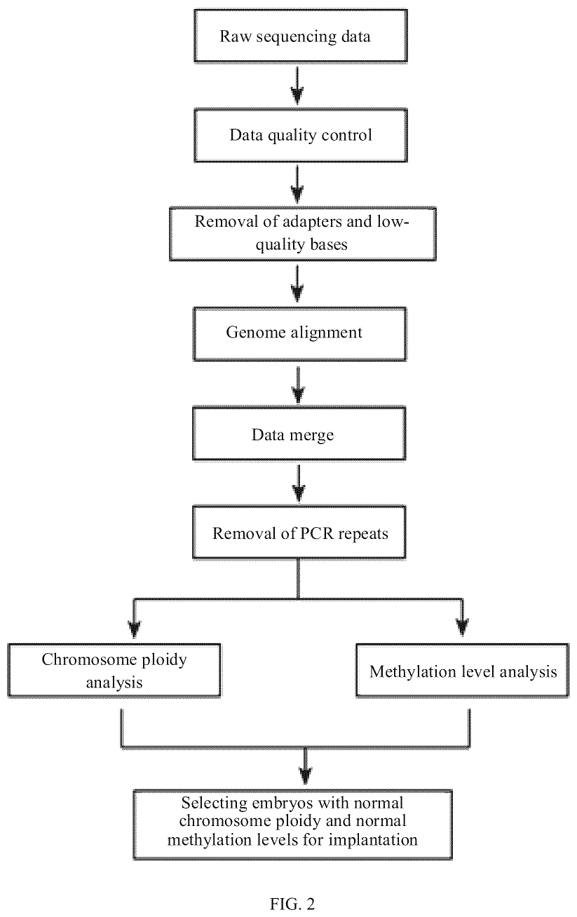

After sequencing, the sequencing data were analyzed according to the data analysis process shown in FIG. 2. For the analysis of the methylation level, in addition to the genome-wide DNA methylation level of the embryos, the methylation level of the imprinted gene control region or/and the gene promoter region was considered.

### 2.2 Testing of Chromosomes of Pre-Implantation Embryos

The current PGS analysis process was adopted to test and analyze chromosomes.

2.3 Testing of DNA methylation level of pre-implantation embryos

For clinically tested embryos, embryos were screened based on the genome-wide DNA methylation level, in which the interval of 0.25 to 0.27 is the optimal implantation standard, and the embryo has the genome-wide DNA methylation level outside the interval of 0.25 to 0.27 but close to the interval of 0.25 to 0.27 relative to other embryos is taken as the embryo to be implanted. Besides, the embryos were also screened based on the methylation level of the imprinted gene control region or/and the gene promoter region to reduce the proportion of birth defects.

### 3. Embryo Implantation

Euploid preimplantation embryos were selected for implantation, and other embryos were frozen according to conventional methods, so if the implantation failed, the patient would still have implantable euploid embryos for subsequent implantation. After implantation, the pregnancy rate, abortion rate and birth rate of embryos with different DNA methylation levels were calculated through the follow-up investigation of the implanted embryos. If the patient had no more euploid embryos to be implanted, the patient should decide whether to implant chimeric embryos after informed of the risk of implantation of chimeric embryos.

In the present application, nearly 250 pre-implantation embryos from 180 voluntary families were numbered, and the methylation level and the aneuploidy were tested according to the above method. The test results were as below.

The statistical results of the pregnancy rate are shown in FIG. 3. It can be seen from FIG. 3 that:
(1) Among the 3 pre-implantation embryos with the genome-wide DNA methylation level less than 0.19, there was one case of implanted pregnancy, with the pregnancy rate of about 33%.
(2) Among the 15 pre-implantation embryos with the genome-wide DNA methylation level between 0.19 to 0.21, there were 9 cases of implanted pregnancy, with the pregnancy rate of about 60%.
(3) Among the 34 pre-implantation embryos with the genome-wide DNA methylation level between 0.22 to 0.24, there were 21 cases of implanted pregnancy, with the pregnancy rate of about 62%.
(4) Among the 43 pre-implantation embryos with the genome-wide DNA methylation level between 0.25 to 0.27, there were 33 cases of implanted pregnancy, with the pregnancy rate of about 77%.
(5) Among the 24 pre-implantation embryos with the genome-wide DNA methylation level between 0.28 to 0.29, there were 16 cases of implanted pregnancy, with the pregnancy rate of about 67%.
(6) Among the 30 pre-implantation embryos with the genome-wide DNA methylation level between 0.30 to 0.33, there were 19 cases of implanted pregnancy, with the pregnancy rate of about 63%.
(7) Among the 9 pre-implantation embryos with the genome-wide DNA methylation level more than 0.34, there were 4 cases of implanted pregnancy, with the pregnancy rate of about 44%.

It can be seen from FIG. 3 that when the genome-wide DNA methylation level is closer to the interval of about 0.25 to 0.27, the corresponding pregnancy rate is higher.

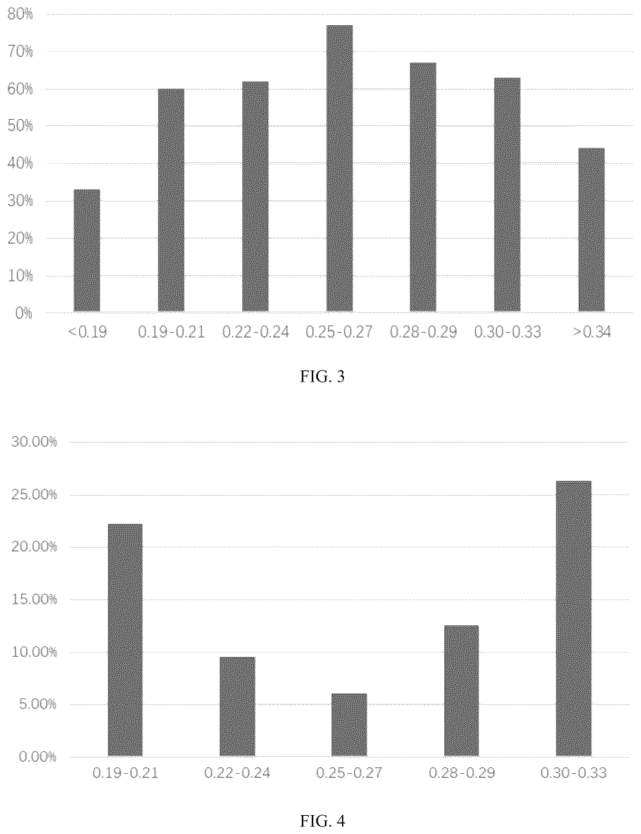

The abortion rate of the pregnant patients shown in FIG. 3 was further tracked and calculated. The results are shown in FIG. 4. It can be seen from FIG. 4 that when the genome-wide DNA methylation level is closer to the interval of about 0.25 to 0.27, the corresponding abortion rate is lower and accordingly the birth rate is higher.

In the above, the embryos for in vitro fertilization were screened based on the genome-wide DNA methylation level of the trophoblast cells. For the same family, if there is an embryo with a methylation level of about 0.25 to 0.27, the embryo is selected for implantation. If there is no embryo with a methylation level within this interval, the embryo which has a genome-wide DNA methylation level within an interval of about 0.22 to 0.24 is in a second implantation priority. The embryo with the genome-wide DNA methylation level within an interval of about 0.28 to 0.29 is in a third implantation priority. The embryo with the genome-wide DNA methylation level within an interval of about 0.19 to 0.21 is in a fourth implantation priority. The embryo with the genome-wide DNA methylation level within an interval of about 0.30 to 0.33 is in a fifth implantation priority.

When a plurality of embryos in a same implantation priority are provided, an embryo is selected as an implantation embryo from the plurality of embryos according to a preferential selecting priority shown below.
the embryo with the lowest absolute difference value between the genome-wide DNA methylation level and 0.26 is preferentially implanted.

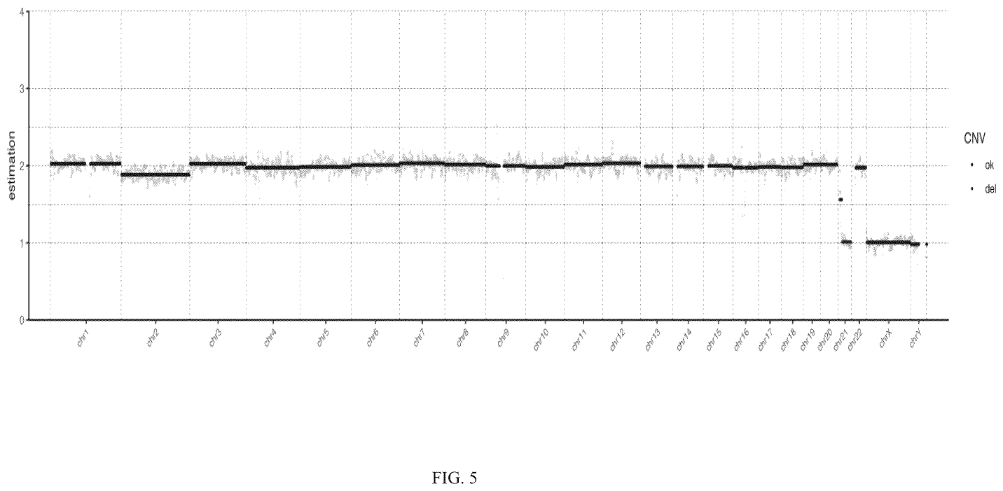

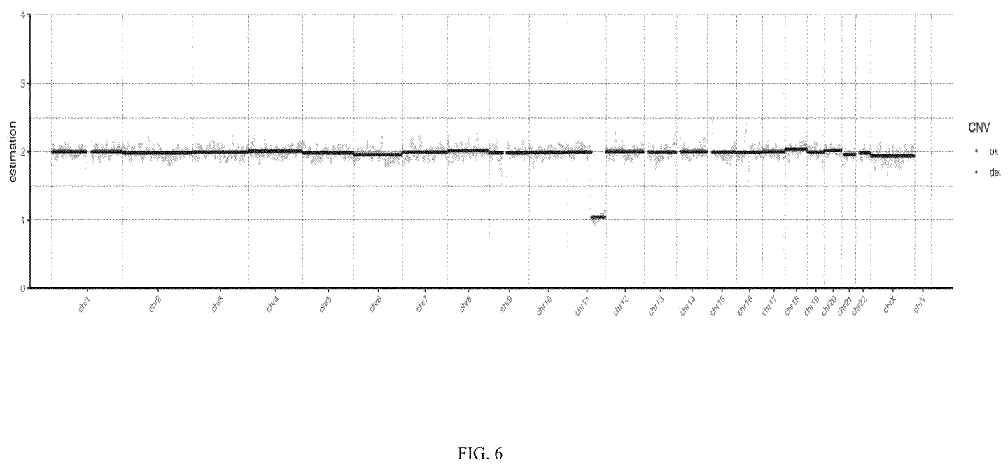

The chromosomal conditions of pre-implantation embryos from some families obtained by analyzing with the method of the present application are shown in FIG. 5 and FIG. 6.

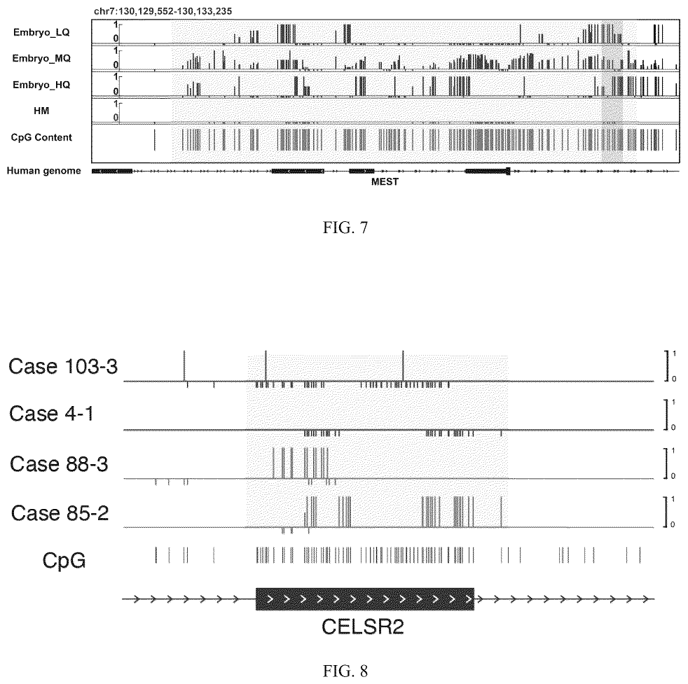

In FIG. 7, MEST is a maternally imprinted gene that plays a key role in embryonic development. FIG. 7 shows a comparison diagram of the methylation level of the EMST gene in healthy-born embryos and abnormally developed embryos. HM represents an abnormally developed embryo. Embryo_HQ, Embryo_MQ and Embryo_LQ represent healthy-born embryos. The shaded part in the figure represents the control region of the gene. It is found that this region has a relatively high methylation level in the born embryos, with the black vertical lines indicating methylation levels, whereas this region is not methylated in the abnormally developed embryo (HM).

In FIG. 8, the CELSR2 gene plays an important role in the differentiation of the ectoderm. FIG. 8 shows the methylation level of a region of the gene in different types of embryos. Case 103-3 and Case 4-1 represent two healthy-born embryos. Case 88-3 and Case 85-2 represent two embryos suffering from pregnancy failures. It is found from FIG. 8 that this region shows low methylation levels in the healthy-born embryos, whereas this region shows a relatively high methylation level in embryos suffering from pregnancy failures.

### Case 1 of Embryo Implantation

The volunteer family in this case possesses four preimplantation embryos. The embryos No. 1 and No. 3 have normal chromosomes, but the embryo No. 3 has an abnormal methylation level, specifically more than 0.34. For the first embryo implantation, the clinician implanted the pre-implantation embryo No. 3, which resulted in pregnancy failure. For the second embryo implantation, the clinician selected the pre-implantation embryo No. 1 which has a normal methylation level within the interval of 0.25 to 0.27, and a healthy child was born.

TABLE 4
Case 1 of embryo implantation

| | Embryo | Genome-wide DNA | | |
|---|---|---|---|---|
| | number | methylation level | Chromosome status | Pregnancy/Birth |
| Case 1 of | 1 | 0.26 | normal chromosome | second time, birth |
| implantation | 2 | 0.24 | abnormal ~22 (mosaic) | |
| | | | (50%) | |
| | 3 | 0.55 | normal chromosome | first time, failed |
| | 4 | 0.31 | abnormal: del( 1) | |
| | | | (p21.3-p36.33) | |

### Case 2 of Embryo Implantation

The volunteer family in this case possesses two pre-implantation embryos. The two pre-implantation embryos have normal chromosomes, but the embryo No. 1 has an abnormal methylation level, specifically less than 0.19. For the first embryo implantation, the clinician implanted the preimplantation embryo No. 1, which resulted in pregnancy failure. For the second embryo implantation, the clinician implanted the pre-implantation embryo No. 2 which has a normal methylation level close to the interval of 0.25 to 0.27, and a healthy child was born.

TABLE 5
Case 2 of embryo implantation

| | | Genome-wide | | |
|---|---|---|---|---|
| | Embryo | DNA methyl- | Chromosome | Pregnancy/ |
| | number | ation level | status | Birth |
| Case 2 of | 1 | 0.16 | normal | first time, |
| implantation | | | chromosome | failed |
| | 2 | 0.24 | normal | second time, |
| | | | chromosome | birth |

The technical features of the above embodiments can be combined arbitrarily. In order to make the description concise, not all possible combinations of the technical features are described in the above embodiments. However, as long as there is no contradiction in the combination of these technical features, the combinations should be considered as within the scope of this specification.

The above-described embodiments are only several implementations of the present application, and the descriptions are relatively specific and detailed, but they should not be construed as limiting the scope of the present application. It should be understood by those of ordinary skill in the art that various modifications and improvements can be made without departing from the concept of the present application, and all fall within the protection scope of the present application. Therefore, the patent protection of the present application shall be defined by the appended claims.

1. A method for screening pre-implantation embryos, comprising: testing chromosomes and DNA methylation levels of the pre-implantation embryos, and selecting, from the pre-implantation embryos according to the obtained test result, the embryo having normal chromosomes, and a genome-wide DNA methylation level within an interval of about 0.25 to 0.27 or a genome-wide DNA methylation level outside the interval of about 0.25 to 0.27 but close to the interval of about 0.25 to 0.27 relative to other embryos as the embryos to be implanted.

2. The method for screening pre-implantation embryos according to claim 1, wherein the pre-implantation embryos are embryos that have developed in vitro for about 5 to 7 days after fertilization.

3. The method for screening pre-implantation embryos according to claim 2, wherein the pre-implantation embryos are embryos at a blastocyst stage.

4. The method for screening pre-implantation embryos according to claim 1, wherein a sample for testing the chromosomes and the DNA methylation level comprises a trophoblast cell from the pre-implantation embryo.

5. The method for screening pre-implantation embryos according to claim 1, wherein the normal chromosomes comprise chromosomes with a normal number and a normal structure, or chromosomes that belong to a chimaera with a degree of chimerism less than about 50%.

6. The method for screening pre-implantation embryos according to claim 1, wherein under the condition that a plurality of embryos to be implanted from a same patient are provided, selecting and implantation priorities for the embryos are as follows:
the embryo with the genome-wide DNA methylation level within an interval of about 0.25 to 0.27 being in a first implantation priority;
the embryo with the genome-wide DNA methylation level within an interval of about 0.22 to 0.24 being in a second implantation priority;
the embryo with the genome-wide DNA methylation level within an interval of about 0.28 to 0.29 being in a third implantation priority;
the embryo with the genome-wide DNA methylation level within an interval of about 0.19 to 0.21 being in a fourth implantation priority; and
the embryo with the genome-wide DNA methylation level within an interval of about 0.30 to 0.33 being in a fifth implantation priority.

7. The method for screening pre-implantation embryos according to claim 6, wherein under the condition that a plurality of embryos in a same implantation priority are provided, an embryo is selected as an implantation embryo from the plurality of embryos in the following preferential selecting and implantation priority:
the embryo with the lowest absolute difference value between the genome-wide DNA methylation level and 0.26 is preferentially implanted.

8. The method for screening pre-implantation embryos according to claim 1, wherein testing the chromosomes comprises pre-implantation genetic screening.

9. The method for screening pre-implantation embryos according to claim 1, further comprising screening the embryos according to the methylation level of an imprinted gene control region or/and a gene promoter region to reduce a proportion of birth defects.

10. The method for screening pre-implantation embryos according to claim 1, wherein testing the DNA methylation level comprises constructing a methylation library and sequencing.
